(19) United States
(12) Patent Application Publication
(10) Pub. No.: US 2012/0077934 A1
(43) Pub. Date: Mar. 29, 2012

(54) Functional Polyisobutylene Based Macromonomers And Methods For Making And Using The Same

(75) Inventors: Rudolf Faust, Lexington, MA (US); Ranjan Tripathy, Lowell, MA (US); Umaprasana Ojha, Lowell, MA (US)

(73) Assignee: University of Massachusetts

(21) Appl. No.: 13/257,220

(22) PCT Filed: Mar. 23, 2010
(86) PCT No.: PCT/US2010/028334

§ 371 Date: Dec. 5, 2011

Related U.S. Application Data
(60) Provisional application No. 61/210,761, filed on Mar. 23, 2009.

(51) Int. Cl. C08F 8/08; C07C 69/602; C07C 41/14; C07C 43/16; C07D 303/40; C07C 67/04; C08F 8/00; C07D 301/00

(52) U.S. Cl. 525/122; 525/312; 525/308; 560/201; 549/539; 568/673; 549/554

(57) ABSTRACT

A method of synthesizing a functionalized polymer represented by the structural formula (I) comprising a step of reacting a polymer represented by structural formula (II) with a compound Nu¹-M to nucleophilically substitute moiety X¹ with moiety Nu¹. Values and preferred values of the variables in formulas (I) and (II) are defined herein.

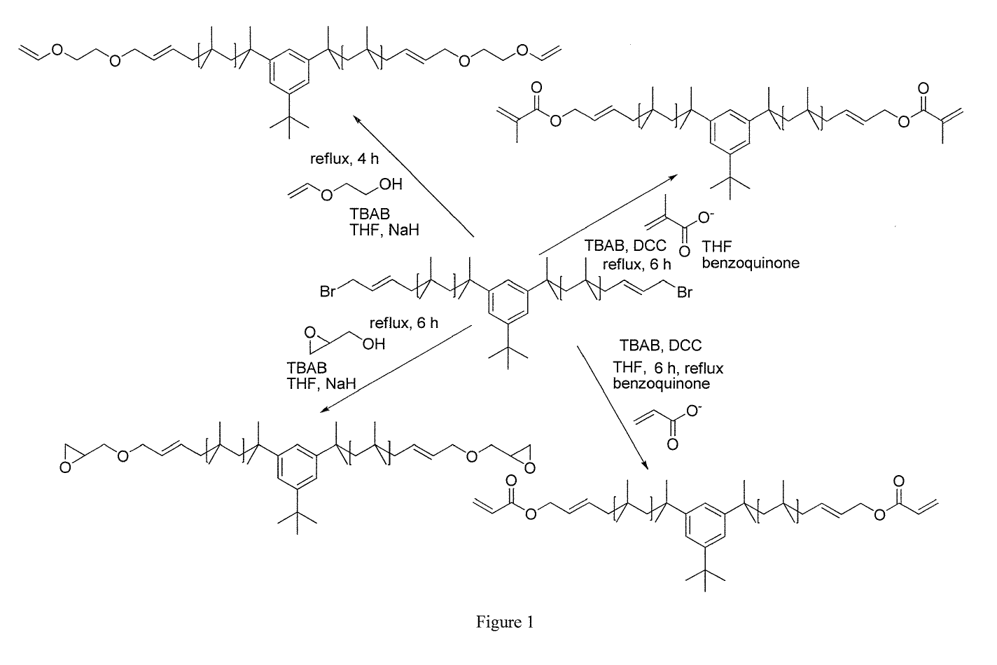

### RELATED APPLICATION

This application claims the benefit of U.S. Provisional Application No. 61/210,761, filed on Mar. 23, 2009. The entire teachings of the above application is incorporated herein by reference.

### GOVERNMENT SUPPORT

The invention was supported, in whole or in part, by a grant CHE-0548466 from the National Science Foundation. The Government has certain rights in the invention.

### BACKGROUND OF THE INVENTION

Functional polymers are of great interest due to their potential applications in many important technological areas such as surface modification, adhesion, drug delivery, compatibilization of polymer blends, motor oil additives, low molecular weight precursors to high polymers, use as polymeric macroinitiators, etc.

A special class of functional polymers called macromonomers, which are the subject of this invention, contain polymerizable end functionalities.

In addition to the controlled and uniform size of the polymers, living polymerizations provide the simplest and most convenient method for the preparation of functional polymers. Although varieties of end-functionalized polymers have successfully been synthesized in anionic polymerization, there are relatively few end-functionalized polymers (polymers with functional groups selectively positioned at the termini of any given polymeric or oligomeric chain) synthesized by living cationic polymerization of vinyl monomers. There are two basic methods to prepare functional polymers by living cationic polymerization: initiation from functional initiators and termination by functional terminators.

Both have been employed to achieve the above target. However, post-polymerization functionalization is preferred, since in ionic polymerization many unprotected functional groups interfere during the course of polymerization. Furthermore, the functional initiator method requires an efficient coupling/linking agent for the preparation of bi- and multi-functional polymers, which are not readily available. The reported procedures to functionalize the polymers involve multi-step synthetic pathway, often result in incomplete end-functionalization and are expensive. The procedures reported to date are complicated, laborious and expensive and, therefore, not practiced commercially. Accordingly, a need exists for novel methods of preparation of high quality functional polymers that overcome limitations of known methods.

### SUMMARY OF THE INVENTION

In one embodiment, the present invention is a method of synthesizing a functionalized polymer represented by the structural formula (I)

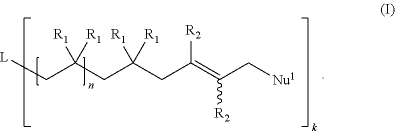

The method comprising a step of reacting a polymer represented by structural formula (II)

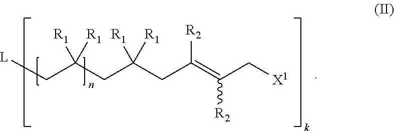

with a compound Nu¹-M to nucleophilically substitute moiety X¹ with moiety Nu¹. In formulas (I) and (II):
n is an integer not less than 2;
k is an integer greater than or equal to 1;
L is absent or is an initiator residue;
R₁ for each occasion is independently H or a C1-C4 alkyl, an alkoxy or a substituted or unsubstituted aryl;
R₂ for each occasion is independently H, X², —CH₂X², —CHX²₂, —CX²₃, —C≡N, or —NO₂;
X¹ and X² are, for each occurrence, independently, a halogen;
M is an alkali metal;
Nu¹ is —Y¹—Y²—R³,

wherein:
Y¹ is absent or is a —NR¹⁰—, —S—, or —O—, wherein R¹⁰ is a C1-C6 alkyl;
Y² is absent or is a C2-C6 alkylene, (—OCH₂CH₂—)₁₋₃, —Si(CH₃)—, or a C2-C6 alkylene-O—;
R³ is a C1-C6 alkyl, functionalized by an epoxy, a thriirane, acrylate, methacrylate, cyano acrylate, a vinyloxy or 4,5-dihydrooxazole moiety.

In another embodiment, the present invention is a functionalized polymer represented by structural formula (I)

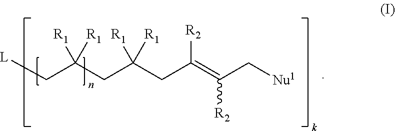

Values and preferred values of the variables in formula (I) are defined above.

The invention includes preparation of functional hydrocarbon polymers by nucleophilic substitutions of haloallyl functional polymers. Haloallyl functional polymers, in turn, can be easily and economically prepared by living cationic polymerization, followed by capping with 1,3-butadiene, as disclosed in U.S. patent application Ser. No. 11/400,059, filed on Apr. 7, 2006. The entire teachings of this Application are incorporated herein by reference.

The methods of the present invention advantageously accomplish syntheses of curable macromonomers based on methacrylate, acrylate, vinyloxy and epoxy end-functional polyisobutylenes (PIB) in a single-step nucleophile substitution reactions from a haloallyl telechelic PIB utilizing inexpensive reagents. The end-functional PIBs can be obtained with quantitative end-functionality.

Polyisobutylene polymers are useful for controlled drug delivery matrixes, polymeric surfactants, compatibilizers, surface modifiers, and scaffolds for tissue engineering. For example, networks based on methacryloyl end-capped telechelic PIB possess controlled drug release abilities, acrylate end-capped PIB are useful as microprocessor and medical device coatings, radiation curable coatings based on vinyloxy end-functional PIB exhibited high refractive index, good adhesion, dampening and barrier properties and amino cross-linked polymers based on epoxy functional PIB can find widespread application as chemical resistance coatings and underwater paints.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

The FIGURE depicts one embodiment of a synthetic scheme employed to produce the end-functional polyisobutylenes (PIBs) of the present invention.

### DETAILED DESCRIPTION OF THE INVENTION

A description of example embodiments of the invention follows.

### DEFINITIONS OF TERMS

As used herein, “alkyl” refers to a straight-chain or branched saturated hydrocarbon group. Examples of alkyl groups include methyl (Me), ethyl (Et), propyl (e.g., n-propyl, and isopropyl), butyl (e.g., n-butyl, isobutyl, s-butyl, t-butyl), pentyl groups (e.g., n-pentyl, isopentyl, neopentyl), and the like. A lower alkyl group typically has up to 6 carbon atoms. In various embodiments, an alkyl group has 1 to 6 carbon atoms, and is referred to as a “C1-6 alkyl group.” Examples of C1-6 alkyl groups include, but are not limited to, methyl, ethyl, propyl (e.g., n-propyl and isopropyl), and butyl groups (e.g., n-butyl, isobutyl, s-butyl, t-butyl). A branched alkyl group has at least 3 carbon atoms (e.g., an isopropyl group) and up to 6 carbon atoms, e.g. it is a C3-6 alkyl group, i.e., a branched lower alkyl group. Examples of branched lower alkyl groups include, but are not limited to, isopropyl, isobutyl, sec-butyl, tert-butyl, isopentyl, neopentyl, and tert-pentyl.

As used herein, the term “alkenyl” means a saturated straight chain or branched non-cyclic hydrocarbon having from 2 to 10 carbon atoms and having at least one carbon-carbon double bond. Representative straight chain and branched C2-C10 alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups may be optionally substituted with one or more substituents.

As used herein, the term “alkynyl” means a saturated straight chain or branched non-cyclic hydrocarbon having from 2 to 10 carbon atoms and having at lease one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups may be optionally substituted with one or more substituents.

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. In various embodiments, a cycloalkyl group has 3-6 carbon atoms, and is referred to as a “C3-6 cycloalkyl group.” Examples of C3-6 cycloalkyl groups include, but are not limited to, cyclopropyl, cyclopropylmethyl, cyclopropylethyl, cyclopropylpropyl, cyclobutyl, cyclobutylmethyl, cyclobutylethyl, cyclopentyl, cyclopentylmethyl, cyclohexyl, cyclopentenyl, cyclohexenyl, and cyclohexadienyl groups, as well as their homologs, isomers, and the like.

As used here, the term “alkylene” refers to a divalent alkyl group that has two points of attachment to the rest of the compound. Non-limiting examples of alkylene groups include a divalebt C1-6 groups such as methylene (—CH₂—), ethylene (—CH₂CH₂—), n-propylene (—CH₂CH₂CH₂—), isopropylene (—CH₂CH(CH₃)—), and the like. Alkylene groups may be optionally substituted with one or more substituents. A divalent C1-6 alkyl group can be a straight chain or branched alkyl group, which as a linking group is capable of forming a covalent bond with two other moieties. Examples of a divalent C1-6 alkyl group include, for example, a methylene group, an ethylene group, an ethylidene group, an n-propylene group, an isopropylene group, an isobutylene group, an s-butylene group, an n-butylene group, and a t-butylene group.

As used herein, “alkoxy” refers to an —O-alkyl group wherein the alkyl group may be a straight or branched chain. Examples of alkoxy groups include, but are not limited to, methoxy, ethoxy, propoxy (e.g., n-propoxy and isopropoxy), t-butoxy groups, and the like.

The term “haloalkyl”, as used herein, includes an alkyl substituted with one or more F, Cl, Br, or I, wherein alkyl is defined above.

The term “aryl”, as used herein, refers to a carbocyclic aromatic group. Examples of aryl groups include, but are not limited to phenyl and naphthyl. Examples of aryl groups include optionally substituted groups such as phenyl, biphenyl, naphthyl, phenanthryl, anthracenyl, pyrenyl, fluoranthyl or fluorenyl. Examples of suitable substituents on an aryl include halogen, hydroxyl, C1-C12 alkyl, C2-C12 alkene or C2-C12 alkyne, C3-C12 cycloalkyl, C1-C12 haloalkyl, C1-C12 alkoxy, aryloxy, arylamino or aryl group.

The term “aryloxy”, as used herein, means an “aryl-O—” group, wherein aryl is defined above. Examples of an aryloxy group include phenoxy or naphthoxy groups.

The term “heteroaryl”, as used herein, refers to aromatic groups containing one or more heteroatoms (O, S, or N). A heteroaryl group can be monocyclic or polycyclic, e.g. a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing heteroaryl groups may be C-attached or N-attached (where such is possible). For instance, a group derived from pyrrole may be pyrrol-1-yl (N-attached) or pyrrol-3-yl (C-attached).

Suitable substituents for heteroaryl are as defined above with respect to aryl group.

Suitable substituents for an alkyl, cycloalkyl include a halogen, an alkyl, an alkenyl, a cycloalkyl, a cycloalkenyl, an aryl, a heteroaryl, a haloalkyl, cyano, nitro, haloalkoxy.

Further examples of suitable substituents for a substitutable carbon atom in an aryl, a heteroaryl, alkyl or cycloalkyl include but are not limited to —OH, halogen (—F, —Cl, —Br, and —I), —R, —OR, —CH₂R, —CH₂OR, —CH₂CH₂OR. Each R is independently an alkyl group.

In some embodiments, suitable substituents for a substitutable carbon atom in an aryl, a heteroaryl or an aryl portion of an arylalkenyl include halogen, hydroxyl, C1-C12 alkyl, C2-C12 alkenyl or C2-C12 alkynyl group, C1-C12 alkoxy, aryloxy group, arylamino group and C1-C12 haloalkyl.

In addition, the above-mentioned groups may also be substituted with ═O, ═S, ═N-alkyl.

In the context of the present invention, an amino group may be a primary (—NH₂), secondary (—NHRₚ), or tertiary (—NRₚR_{q}), wherein Rₚ and R_{q} may be any of the alkyl, alkenyl, alkynyl, alkoxy, cycloalkyl, cycloalkoxy, aryl, heteroaryl, and a bicyclic carbocyclic group.

As used herein, a chemical moiety is “functionalized” if it includes a functional group. Examples of polymerizable functional groups include an epoxy group

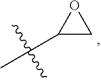

a thriirane group

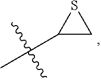

an acrylate group

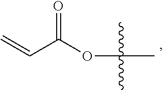

a methacrylate group

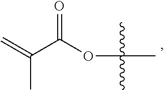

a cyano acrylate group

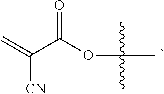

a vinyloxy group

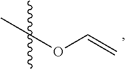

### Or

a 4,5-dihydrooxazole group

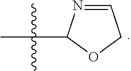

Functional groups can be attached to the functionalized moiety directly or via one or more linkers. Examples of linkers include an alkyl, a —NR*—, —S—, and —O—, where R* is a hydrogen or an alkyl.

As used herein, an “alkali metal” is a metal of Group 1, typically, Li, K or Na. More typically, an alkali metal is K or Na. Even more typically, an alkali metal is Na.

### Synthesis of Haloallyl End-Functional Polymers

In various embodiments, this invention utilizes a method to “cap” a living polyolefin cation, typically a polyisoolefin cation, even more typically a living polyisobutylene cation (PIB⁺), with a capping agent.

The preferred method is described in U.S. patent application Ser. No. 11/400,059, “Capping Reactions in Cationic Polymerization; Kinetic and Synthetic Utility,” filed on Apr. 7, 2006, and incorporated herein by reference in its entirety. As described in the referenced application, selected conditions have been discovered under which termination is faster than propagation of butadiene (kₜ>>kₚ), resulting in carbocations reacting with olefins to yield the [1:1] adduct exclusively. As used herein, the term “faster” means at least 10-fold faster, preferably at least 100-fold faster, and more preferably 1000-fold faster, under otherwise similar conditions.

A capping agent can include optionally substituted olefins, such as optionally substituted conjugated dienes, and optionally substituted butadienes. As another example, unsubstituted butadienes can be employed.

A “living” cationic polyolefin, generally, is any polyolefin with a terminal cationic group and is termed “living” polymers because it is typically made by one of many living polymerization methods known to those of ordinary skill in the art. Generally, living polymerization is a form of addition polymerization where the ability of a growing polymer chain to terminate has been removed. In various embodiments, a polyolefin, e.g., polyisoolefin, polymultiolefin or poly(substituted or unsubstituted vinylidene aromatic compounds), and, more typically polyisobutylene, can be reacted with an optionally substituted conjugated diene, e.g., butadiene, to “cap” the polymer, wherein the cap is halide terminated group. Suitable polyolefins can include C₄ to C₁₈ polyisomonoolefins, C₄ to C₁₄ polymultiolefins, and poly(substituted or unsubstituted vinylidene aromatic compounds), for example C₄ to C₁₀ polyisomonoolefins, or more typically C₄ to C₈ polyisomonoolefins. Polyisobutylene is an example of a preferred isoolefin polymer.

One set of reaction conditions that can produce these polymeric carbocations is, in a solvent, to contact the olefin monomer with an initiating system comprising an initiator (usually an organic ether, organic ester, or organic halide) and a co-initiator. The co-initiator is typically used in concentrations equal to or typically 2 to 40 times higher than the concentration of the initiator. Examples of co-initiators include one or more of BCl₃, TiCl₄, AlBr₃, and organoaluminum halides such as Me₃Al₂Br₃, MeAlBr₂, and Me₂AlBr.

The polymerization can typically be conducted in a temperature range of from about −10° to about −100° C., typically from about −50° to about −90° C. for about 10 to about 120 minutes, depending on the concentration of the initiator and the co-initiator.

Once the desired living polymer is obtained, the capping agent, e.g., optionally substituted butadiene, can be added to the polymerization media in concentrations equal to up to about 10 times the concentration of the living chain ends. The butadiene generally is reacted with the living polymer for about 10 minutes to about 5 hours, depending on the concentration of the living chain ends and the butadiene. The time necessary to achieve essentially 100% capping will vary with the initiator, co-initiator and butadiene concentrations. With higher initiator concentrations the time is shorter, about 20 minutes, while lower initiator concentrations may require 10 hours to achieve 100% capping.

The living polymers employed in the methods of the present invention can be, for example, homopolymers, copolymers, terpolymers, and the like depending upon the olefinic chargestock used. Preferred number average molecular weights (Mn) of the living polymers of the present invention may range from about 500 to about 2,000,000, generally from about 2,000 to about 100,000, or in some embodiments from about 1500 to about 5000. Preferably, the polymers have a narrow molecular weight distribution such that the ratio of weight average molecular weight to number average molecular weight (M_{w}/Mₙ) of the polymers ranges from about 1.0 to about 1.5, and typically from about 1.0 to about 1.2. The polymers can be recovered from the polymerization zone effluent and finished by conventional methods. In one embodiment, synthesizing an end-capped polymer according to the techniques described herein results in a very high yield (up to about 100%) of a functionalized monoaddition product of butadiene to the polymer chain.

In a preferred embodiment, the methods of the present invention employ, as a starting material, a polymer represented by structural formula (II)

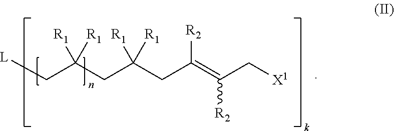

The polymer represented by structural formula (II) is obtained by reacting, in a solvent, a cationic living polymer represented by structural formula (III)

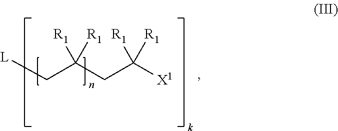

with an optionally substituted conjugated diene represented by structural formula (IV) as an endcapping reagent, in the presence of a Lewis acid,

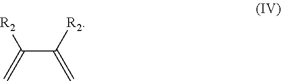

Preferably, the solvent causes termination by halogenation to be faster than the addition of additional molecules of the conjugated diene, thereby producing the endcapped polymer having a halogenated endcap group.

In formulas (II), (III) and (IV):
n is an integer not less than 2;
k is an integer greater than or equal to 1;
L is absent or is an initiator residue;
R₁ for each occasion is independently H or a C1-C4 alkyl, an alkoxy or a substituted or unsubstituted aryl;
R₂ for each occasion is independently H, X², —CH₂X², —CHX²₂, —CX²₃, —C≡N, or —NO₂; and
X¹ and X² are, for each occurrence, independently, a halogen. Preferably, X¹ and X² are, for each occurrence, independently Cl or Br. More preferably, X¹ and X² are, for each occurrence, independently Br.

As used herein, a substituent on a carbon atom that forms an unsaturated carbon-carbon bond and whose attachment to such carbon atom is denoted by the symbol [symbol] can be in either cis or trans substituent. The remainder of values and preferred values for the variable in formulas (II) and (III) are as defined above.

As used herein, an “initiator residue” (L) is a chemical moiety linking k polymeric moieties in formulas (II) or (III). A linking moiety can include cumyl, dicumyl and tricumyl when cumyl, dicumyl or tricumyl chloride, methylether or ester is used as initiator. Other examples include 2,4,4,6-tetramethylheptylene or 2,5-dimethylhexylene, which arise when 2,6-dichloro-2,4,4,6-tetramethylheptane or 2,5-dichloro-2,5-dimethylhexane is used as initiator. Preferably, L is 5-tert-butyl-dicumyl, 1,3,5-tri-cumyl, 2,4,4,6-tetramethylheptyl and 2,5-dimethylhex-3-en-yl. Many other cationic mono- and multifunctional initiators are known in the art. One skilled in the art will be able to select suitable initiator residues.

In one embodiment of the polymer represented by structural formula (II), k is 2, and L is represented by the following structural formula

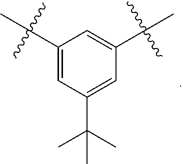

Preferably, polymer of formula (II) is represented by the following structural formula:

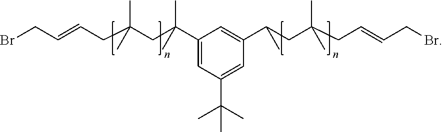

Preferably, the cationic living polymer represented by structural formula (III) is obtained by reacting a cationically polymerizable monomer in the presence of a coinitiator. Typically, the coinitiator is one or more of BCl₃, TiCl₄, and organoaluminum halides. In various embodiments, the reaction proceeds in a solvent that comprises at least one component having a dielectric constant less than about 9. Typically, the solvent is selected from one or more of hexane, cyclohexane, methylcyclohexane, methylchloride, n-butyl chloride, dichloromethane, toluene, and chloroform.

Solvents suitable for practicing the reactions described above are, for example, solvents that include at least one component having a dielectric constant less than 9. Preferably, the solvents include at least one component having a dielectric constant less than 7. Alternatively, the solvents include a mixture of at least one solvent having a polar solvent with a dielectric constant equal to or higher than 9 and at least one nonpolar solvent with a dielectric constant lower than 6. Examples of suitable solvents include one or more of hexane, cyclohexane, methylcyclohexane, methylchloride, n-butyl chloride, dichloromethane, toluene, and chloroform.

### Nucleophilic Substitution of Haloallyl End-Functional Polymers

In one embodiment, the present invention is a method of synthesizing a functionalized polymer represented by the structural formula (I)

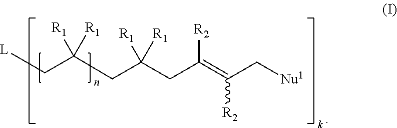

The method comprises a step of reacting a polymer represented by structural formula (II)

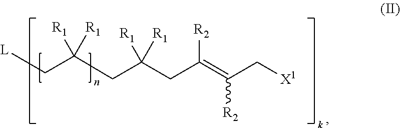

with a compound Nu¹-M to nucleophilically substitute moiety X¹ with moiety Nu¹.

The values of variables in structural formula (I) are as defined above with respect to structural formula (II). For convenience, these values are reproduced below:
n is an integer not less than 2;
k is an integer greater than or equal to 1;
L is absent or is an initiator residue;
R₁ for each occasion is independently H or a C1-C4 alkyl, an alkoxy or a substituted or unsubstituted aryl;
R₂ for each occasion is independently H, X², —CH₂X², —CHX²₂, —CX²₃, —C≡N, or —NO₂; and
X¹ and X² are, for each occurrence, independently, a halogen.
Additionally, M is an alkali metal and Nu¹ is —Y¹—Y²—R³. In formula (I):
Y¹ is absent or is a —NR¹⁰—, —S—, or —O—, wherein R¹⁰ is a C1-C6 alkyl;
Y² is absent or is a C2-C6 alkylene, (—OCH₂CH₂—)₁₋₃, —Si(CH₃)₂—, or a C2-C6 alkylene-O—;
R³ is a C1-C6 alkyl, functionalized by an epoxy, a thriirane, acrylate, methacrylate, cyano acrylate, a vinyloxy or 4,5-dihydrooxazole moiety.

In one embodiment, Y¹ is absent or is —O—. Values and preferred values of the remainder of the variables are as defined herein with respect to structural formulas (I) and (II).

In another embodiment, Y² is absent or is a C2-C6 alkylene, or a C2-C6 alkylene-O—. Alternatively, Y² is (—OCH₂CH₂—)₁₋₃ or —Si(CH₃)₂—. Values and preferred values of the remainder of the variables are as defined herein with respect to structural formulas (I) and (II).

In another embodiment, R³ is a C1-C6 alkyl, functionalized by an epoxy, acrylate, methacrylate, or a vinyloxy moiety. Values and preferred values of the remainder of the variables are as defined herein with respect to structural formulas (I) and (II).

Preferably, Y¹ is —O—, Y² is absent or is a C2-C6 alkylene or a C2-C6 alkylene-O—, and R³ is a C1-C6 alkyl, functionalized by an epoxy or a vinyloxy moiety. Values and preferred values of the remainder of the variables are as defined herein with respect to structural formulas (I) and (II).

In another embodiment, in formula (I), Y¹ is absent, and Y² is absent or is a —C1-C6 alkylene or (—OCH₂CH₂—)₁₋₃, and R³ is selected from methacrylate, acrylate, and cyano acrylate. Preferably, Y² is a —C1-C6 alkylene and R³ is selected from methacrylate, acrylate. Values and preferred values of the remainder of the variables are as defined above with respect to structural formulas (I) and (II).

In one embodiment, Nu¹ is selected from —O—R^{a}—O—CH═CH₂, wherein R^{a} is a C2-C6 alkylene, and —R^{b}—OC(O)C(R^{c})═CH₂, wherein R^{b} is absent or is —OCH₂CH₂— and R^{c} is —H, —CN or —CH₃. Values and preferred values of the remainder of the variables are as defined above with respect to structural formulas (I) and (II).

In another embodiment, Nu¹ is selected from —O—R^{d}, wherein R^{d} is an epoxy-functionalized C1-C6 alkyl, and

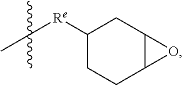

wherein R^{e} is a —O—C2-C6 alkylene, (—OCH₂CH₂—)₁₋₃, or —O—Si(CH₃)₂—CH₂—. Preferably, Nu¹ is

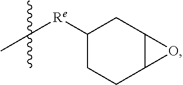

and
R^{e} is a —O—C2-C6 alkylene. Values and preferred values of the remainder of the variables are as defined above with respect to structural formulas (I) and (II).

In another embodiment, Nu¹ is selected from

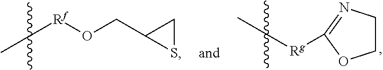

wherein R^{f} is a —O—C2-C6 alkylene, or (—OCH₂CH₂—)₁₋₃, and R^{g} is a —O—C1-C6 alkylene. Values and preferred values of the remainder of the variables are as defined above with respect to structural formulas (I) and (II).

In one embodiment, Nu¹ is —O—R^{a}—O—CH═CH₂, and R^{a} is a C₂-C₆ alkylene. Preferably, Nu¹ is —O—(CH₂)₂—O—CH═CH₂. Values and preferred values of the remainder of the variables are as defined above with respect to structural formulas (I) and (II).

In another embodiment, Nu¹ is —R^{b}—OC(O)C(R^{c})═CH₂, R^{b} is absent or is —OCH₂CH₂— and R^{e} is H, —CN or —CH₃. Preferably, Nu¹ is —OC(O)CH═CH₂ or —OC(O)C(CH₃)═CH₂. Values and preferred values of the remainder of the variables are as defined above with respect to structural formulas (I) and (II).

In another embodiment, Nu¹ is —O—R^{d}, and R^{d} is a 1,2-epoxy-(C1-C6)alkylene. Preferably, Nu¹ is 1,2-epoxy-1-propoxy group. Values and preferred values of the remainder of the variables are as defined above with respect to structural formulas (I) and (II).

In another embodiment, R₁ for each occasion is independently H or a C1-C4 alkyl, and R₂ for each occasion is independently H, X², —CH₂X², —CHX²₂, —CX²₃. Preferably, X² is Cl or Br. Values and preferred values of the remainder of the variables are as defined above with respect to structural formulas (I) and (II).

In another embodiment, X¹ is Cl or Br. Preferably, X¹ is Br. Values and preferred values of the remainder of the variables are as defined above with respect to structural formulas (I) and (II).

In preferred embodiment, the nucleophilic substitution moiety X¹ with moiety Nu¹ takes place in the presence of _tetra-n-butyl ammonium bromide (TBAB) or 1,4,7,10,13,16-hexaoxacyclooctadecane (18-crown-6) and in presence or absence of 1,4-benzoquinone.

In preferred embodiment, the method of synthesizing a functionalized polymer represented by the structural formula (I) further includes a step of producing the polymer represented by structural formula (II)

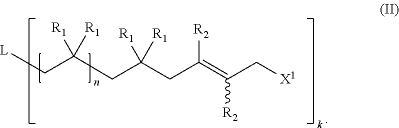

The polymer of represented by formula (II) is obtained by reacting, in a solvent, a cationic living polymer represented by structural formula (III)

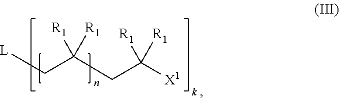

with an optionally substituted conjugated diene represented by structural formula (IV) as an endcapping reagent, in the presence of a Lewis acid,

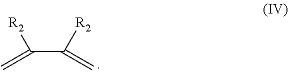

Typically, the solvent causes termination by halogenation to be faster than the addition of additional molecules of the conjugated diene, thereby producing the endcapped polymer having a halogenated endcap group.

In further embodiments, the method of synthesizing a functionalized polymer represented by the structural formula (I) further including the step of producing the cationic living polymer represented by structural formula (III) by reacting a cationically polymerizable monomer in the presence of a coinitiator. Examples of a coinitiator include one or more of BCl₃, TiCl₄, and organoaluminum halides. Preferably, the solvent comprises at least one component having a dielectric constant less than about 9. For example, the solvent can be at least one member selected from the group consisting of hexane, cyclohexane, methylcyclohexane, methylchloride, n-butyl chloride, dichloromethane, toluene, and chloroform.

In certain embodiments, the method of synthesizing a functionalized polymer represented by the structural formula (I) employs an initiator residue L selected from 5-tert-butyl-dicumyl, 1,3,5-tri-cumyl, 2,4,4,6-tetramethylheptyl, 2,5-dimethylhex-3-en-yl. Values and preferred values of the remainder of the variables are as defined above with respect to structural formulas (I) and (II).

In preferred embodiments of the method of synthesizing a functionalized polymer represented by the structural formula (I), k is 2, and L is represented by the following structural formula

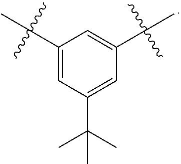

Values and preferred values of the remainder of the variables are as defined above with respect to structural formulas (I) and (II).

Preferably, the polymer of formula (II) is represented by structural formula (IX):

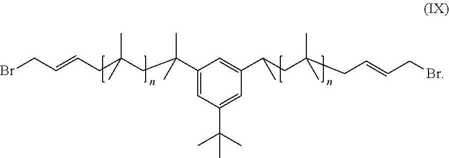

Values and preferred values of the remainder of the variables are as defined above with respect to structural formulas (I) and (II).

More preferably, in the method of synthesizing a functionalized polymer represented by the structural formula (I), Nu¹ is represented by the following structural formula

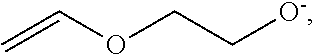

and
the polymer of formula (I) is represented by structural formula (V):

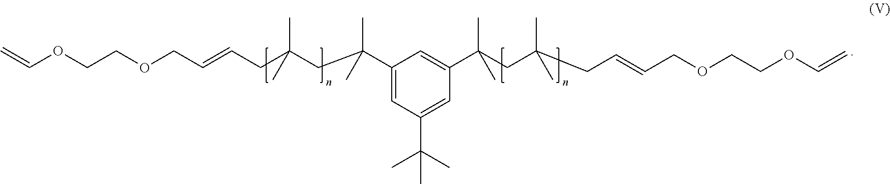

Alternatively, in the method of synthesizing a functionalized polymer represented by the structural formula (I), Nu¹ is represented by the following structural formula

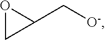

and
the polymer of formula (I) is represented by structural formula (VI):

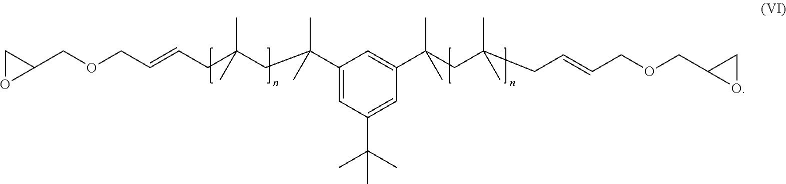

In yet another embodiment of the method of synthesizing a functionalized polymer represented by the structural formula (I), Nu¹ is represented by the following structural formula

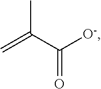

and
the polymer of formula (I) is represented by structural formula (VII):

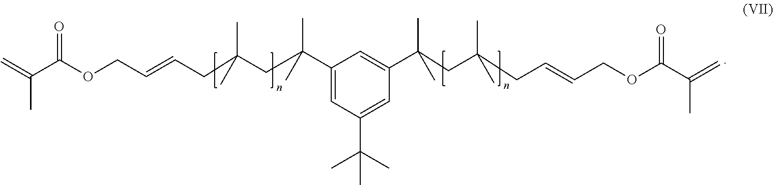

Alternatively, in the method of synthesizing a functionalized polymer represented by the structural formula (I), Nu¹ is represented by the following structural formula

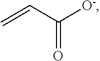

and
the polymer of formula (I) is represented by structural formula (VIII):

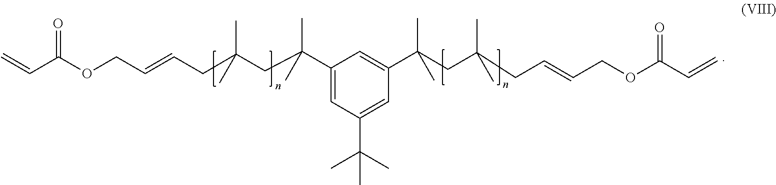

Typical conditions for synthesizing a functionalized polymer represented by the structural formula (I) are as follows. The reaction is carried out in a solvent, for example THF, in a temperature range of 25° C. to 65° C. the reactions were carried out under nitrogen or argon atmosphere, preferably under reflux. A solubilizing agent, e.g. TBAB or 18-crown-6 can be utilized where necessary.

### EXEMPLIFICATION

### Synthesis of Methacrylate, Acrylate, Vinyloxy and Epoxy End-Functional Polyisobutylenes

The precursor bromo end-functional polyisobutylene (PIB) (Mₙ=1200, PDI=1.09) was synthesized by halogen exchange from chloro end-functional PIB (Mₙ=1100, PDI=1.09) as reported before. The end-functional PIBs were synthesized according to Scheme 1 shown in the FIGURE.

### Materials

Sodium methacrylate (Aldrich, 99%), sodium acrylate (Aldrich, 97%), glycidol (Aldrich, 96%), ethylene glycol vinyl ether (Aldrich, 97%), N,N-dicyclohexylcarbodimide (DCC Alfa Aesar, 99%), TBAB (Aldrich, 99%), 18-crown-6 (Aldrich, 99%), sodium hydride (NaH Aldrich, 60% dispersion in mineral oil), potassium hydroxide (KOH, Aldrich), sodium sulfate (Na₂SO₄, Aldrich) and 1,4-benzoquinone (Aldrich, 98%) were used as received. Tetrahydrofuran (THF, Aldrich, 99%) was refluxed over sodium metal and benzophenone over night and distilled under nitrogen atmosphere prior to use.

### Measurements

¹H and ¹³C NMR spectroscopy for structural analysis was carried out on a Bruker 500 MHz spectrometer using CDCl₃ (Cambridge Isotope laboratories, Inc.) as a solvent. ¹H or ¹³C NMR spectra of solution in CDCl₃ were calibrated to tetramethylsilane (TMS) as internal standard (δ H or δ C 0.00). The absolute molecular weight were measured with a Waters HPLC system equipped with a model 515 HPLC pump, model 2410 differential refractometer (λ=930 nm), model 2487 absorbance detector (λ=254 nm), online MALLS detector (MiniDawn, Wyatt Technology Inc., 120 V, three angles; 45°, 90°, and 135°, λ=690 nm), model 712 sample processor, and five Ultrastyragel GPC columns connected in the following series: 500, 10³, 10⁴, 10⁵, and 100 Å. THF was used as eluant at a flow rate of 1.0 mL/min at room temperature. Mₙ and polydispersity index (PDI) data were calculated based on MALLS and RI with the ASTRA 5.3 software (Wyatt Technology Inc.). Matrix-assisted laser desorption/ionization time-of-flight mass spectrometry (MALDI-TOF-MS) was carried out on a micromass M@LDI MALDI-TOF-MS (Waters Corp.) equipped with a 337 nm N₂ laser for end group analysis. All spectra were obtained in the positive ion mode using an accelerating voltage of 15 kV and low laser frequency. The sample was recorded in linear mode, and the average mass of each peak is reported to a Dalton. The source voltage and delay time were optimized to achieve maximum signal to noise ratio. External calibration was performed using polystyrene standards (Mₙ=2000 and 1600 Da, Polysciences, Inc.). The matrix solution was prepared by dissolving dithranol in THF at a concentration of 20 mg/mL. CF₃COOAg (AgTFA) was dissolved in THF (10 mg in 1 mL) to prepare the stock solution. The polymer solution was made by dissolving 10 mg in 1 mL of THF. The polymer, dithranol, and AgTFA solution were mixed in 10:10:1 volume ratio, and 1 μL of the resulting solution was evaporated on the sample holder. The rate of photo-polymerizations was investigated using Optical Pyrometer (OP) apparatus. It consists of an Omega OS552-V1-6 Industrial Infrared Thermometer (Omega Engineering, Inc., Stamford, Conn.) equipped with a laser-sighting device (0S550-LS) mounted at the top of a 40 cm×35 cm×35 cm acrylic irradiation chamber. The focal point of the sensor of the OP instrument can be adjusted using the laser aligner. UV light was supplied to the sample chamber via a UVEX Model SCU-110 mercury lamp equipped with a 95 cm liquid light pipe and directed onto the sample stage at a 45° angle. Liquid light pipe selectively allow light of wavelengths greater than 300 nm to irradiate the sample stage. The light intensity was modified by using different light pipe or by using a mesh to shield the light intensity. The intensity of UV irradiation was measured with a UV Process Supply Inc Control Cure Radiometer. The sample stage consists of a platform that was milled to accept a 2 cm×2 cm plastic slide frame. Hence, it provides a way for fixing the position of sample in the irradiation chamber. Temperature data was collected at a rate of 10 measurements per second and directly recorded and downloaded to an IBM 350-P137 personal computer for analysis. Mechanical properties (flexural modulus and flexural strength) were measured at room temperature (25° C.) and atmospheric conditions on an Instron Model 4400R. All tests were carried out according to ASTM E790.

### Example 1

### Synthesis of Methacrylate End-Functional Polyisobutylene

Bromo end-functional PIB (Mₙ=1200, PDI=1.09, 200 mg, 0.166 mmol) was dissolved in dry THF (5 mL) and was added into a two necked glass reactor followed by the addition of sodium methacrylate (0.045 mg, 0.42 mmol), TBAB (135 mg, 0.42 mmol) and 1,4 benzoquinone (to prevent homopolymerization of end group) (5 mg, 0.005 mmol); the mixture was heated at 65° C. under a dry nitrogen atmosphere for 6 h. The reaction mixture was cooled to room temperature, and THF was evaporated using rotary vacuum evaporator. The residue was dissolved in hexane and the solution was filtered and the filtrate (polymer solution) was precipitated in methanol. The polymer was allowed to settle down at the bottom. The supernatant liquid was decanted off and the sticky mass was dried under vacuum at room temperature for 12 h. Gravimetric yield: 98%, GPC-MALLS: Mₙ=1500, PDI=1.11. ¹H NMR (CDCl₃, ppm, δ): 4.6 (d, 2H, CH₂OCOCH₃C═CH₂), 5.65 (m, 1H, —CH═CHCH₂OCOCH₃C═CH₂), 5.85 (m, 1H, —CH═CHCH₂OCOCH₃C═CH₂), 5.6 and 6.15 (s, 2H, —CH₂), 2.0 (s, 3H—CH₃C═CH₂). ¹³C NMR (CDCl₃, ppm, δ): 167 (OCOCH₃C═CH₂), 149 (═C, Ar), 148.5 (CCH, Ar), 137 (—CH₃C═CH), 133.5 (—CH═CHCH₂OCOCH₃C═CH₂), 126.5 (—CH₃C═CH₂), 125 (CH═CH CH₂OCOCH₃ C═CH₂), 121 (—CH═C, Ar), 120 (—CH═C, Ar), 65.5 (CH₂OCOCH₃C═CH₂).

### Example 2

### Synthesis of Acrylate End-Functional Polyisobutylene

Bromo end-functional PIB (Mₙ=1200, PDI=1.09, 200 mg, 0.166 mmol) was dissolved in dry THF (5 mL) and was added into a two necked glass reactor followed by the addition of sodium acrylate (0.045 mg, 0.42 mmol), TBAB (135 mg, 0.42 mmol) and 1,4 benzoquinone (to prevent homopolymerization of end group) (5 mg, 0.005 mmol); the mixture was heated at 65° C. under a dry nitrogen atmosphere for 6 h. The reaction mixture was cooled to room temperature, and THF was evaporated using rotary vacuum evaporator. The residue was dissolved in hexane and the solution was filtered and the filtrate (polymer solution) was precipitated in methanol. The polymer was allowed to settle down at the bottom. The supernatant liquid was decanted off and the sticky mass was dried under vacuum at room temperature for 12 h. Gravimetric yield: 98%, GPC-MALLS: Mₙ=1400, PDI=1.11. ¹H NMR (CDCl₃, ppm, δ): 4.65 (d, 2H, CH₂OCOCH═CH₂), 5.60 (m, 1H, —CH═CHCH₂OCOCH═CH₂), 5.85 (m, 1H, —CH═CHCH₂OCOCH═CH₂). 5.85 (s, 1H, —CH), 6.15 (m, 1H, CH═CH₂), 6.4 (d, 1H, —CH). ¹³C NMR (CDCl₃, ppm, δ): 166 (OCOCH═CH₂), 149 (═C, Ar), 148.5 (C═CH, Ar), 134 (CH═CHCH₂OCOCHC═CH₂), 128.5 (—CH═CH₂), 131 (—CH═CH₂), 126 (CH═CHCH₂OCOCHC═CH₂), 121 (—CH═CH, Ar), 120 (—CH═C, Ar), 65.5 (CH₂OCOCHC═CH₂).

### Example 3

### Synthesis of Epoxy End-Functionl Polyisobutylene

Bromo end-functional PIB (Mₙ=1200, PDI=1.09, 800 mg, 0.66 mmol) was dissolved in dry THF (5 mL). Glycidol (495 mg, 6.6 mmol), NaH (40 mg, 1.65 mmol) and TBAB (1.06 g, 3.3 mmol) were added and the mixture was refluxed under a dry nitrogen atmosphere for 1.5 h. The reaction mixture was cooled to room temperature, and THF was evaporated. The residue was dissolved in hexanes, the solution was filtered and the filtrate was reprecipitated in methanol. The product obtained was further purified by dissolution and reprecipitation using hexanes and methanol. The product polymer was finally dried under vacuum at room temperature. Gravimetric yield: 97%, GPC-MALLS: Mₙ=1300, PDI=1.15. Yield: 95%. ¹H NMR (CDCl₃, ppm, δ): 4.05 (m, 2H, CH₂OCH₂(CHOCH₂)), 5.55 (m, 1H, CH═CHCH₂OCH₂(CHOCH₂)), 5.75 (m, 1H, —CH═CHCH₂OCH₂(CHOCH₂)), 3.7 and 3.4 (d, 2H, —OCH₂(CH₂OCH)), 3.2 (m, 1H, —(CHOCH₂), 2.8 and 2.6 (m, 1H, (CHOCH₂). ¹³C NMR (CDCl₃, ppm, δ): 149 (C═CH, Ar), 148.5 (—C═CH, Ar), 132.5 (CH═CHCH₂OCH₂(CHOCH₂)), 128 (—C═CH, Ar), 121.5 (—CH═CHCHOC(CHOCH₂)), 120(CH═C, Ar), 70 (OCH₂(CHOCH₂)), 72.5 (CH₂OCH₂(CHOCH₂)), 51 (CHOCH₂), 45 (CHOCH₂).

### Example 4

### Synthesis of Vinyloxy End-Functional Polyisobutylene

Bromo end-functional PIB (Mₙ=1200, PDI=1.09, 900 mg, 0.75 mmol) was dissolved in dry THF (5 mL) and charged into two necked glass reactor equipped with a condenser. Ethylene glycol vinyl ether (660 mg, 7.5 mmol), NaH (43 mg, 1.8 mmol), and TBAB (1.2 g, 3.75 mmol) were charged into the reactor under a slow stream of dry nitrogen atmosphere and the mixture was refluxed for 1 h. The reaction mixture was cooled to room temperature, and THF was evaporated. The residue was dissolved in hexanes, the solution was filtered and the filtrate was reprecipitated in methanol. The product was purified as mentioned above. Yield: 95%. GPC-MALLS: Mₙ=1400, PDI=1.1. ¹H NMR (CDCl₃, ppm, δ): 4.05 (d, 3H CH₂(OCH₂CH₂OCH═CH₂), 5.55 (m, 1H, CH═CHCH₂(OCH₂CH₂OCH═CH₂), 5.75 (m, 1H, CH═CHCH₂(OCH₂CH₂OCH═CH₂), 3.7 (m, 2H, CH₂CH₂OCH═CH₂), 3.8 (m, 2H, CH₂OCH═CH₂), 4.2 (d, 1H, CH₂OCH═CH₂), 6.5 (q, 1H, CH₂OCH═CH₂). ¹³C NMR (CDCl₃, ppm, δ): 152.5 (OCH₂CH₂OCH═CH₂), 149 (C═CH, Ar), 148.5 (C═CH, Ar), 132.5 (CH═CHCH₂OCH₂CH₂OCH═CH₂), 128.5 (CH═CHCH₂OCH₂CH₂OCH═CH₂), 121 (—CH═C, Ar), 120 (—CH═C, Ar), ═CH₂), 67.5 (CH₂OCH₂CH₂OCH═CH₂). 86.5 (CH₂OCH═CH₂), 73 (OCH₂ CH₂OCH═CH₂).

### Example 5

### Synthesis of Epoxy End-Functional Polyisobutylene

The synthesis of epoxy end-functional PIB was attempted using a similar procedure as described in Pat. Int. Appl. 2008, WO 2008060333, the entire teachings of which are incorporated herein by reference.

Chloro end-functional PIB (Mₙ=2200, PDI=1.09, 100 mg, 0.045 mmol) was dissolved in dry THF (10 mL) and was added into a two necked glass reactor followed by the addition of Glycidol (167 mg, 2.27 mmol) and KOH (127 mg, 2.27 mmol). The mixture was refluxed under a dry nitrogen atmosphere for 6 hours. The reaction mixture was cooled to room temperature, and THF was evaporated using a rotary vacuum evaporator. The residue was dissolved in hexanes, the solution was filtered and the filtrate (polymer solution) was precipitated in methanol. The polymer was allowed to settle down. The supernatant liquid was decanted and the sticky mass was dried under vacuum. The ¹H NMR spectrum of the product displayed resonances for chloro end-functional PIB only indicating the absence of reaction.

### Example 6

### Synthesis of Methacrylate-Polyisobutylene-Methacrylate

Bromo end-functional PIB (Mₙ=1200, PDI=1.09, 200 mg, 0.166 mmol) was dissolved in dry THF (5 mL) and was added into a two necked glass reactor followed by the addition of sodium methacrylate (0.045 mg, 0.42 mmol), TBAB (135 mg, 0.42 mmol) 18-crown-6 (0.05 mg, 0.21 mmol) and 1,4 benzoquinone (5 mg, 0.005 mmol); the mixture was heated at 65° C. under a dry nitrogen atmosphere for 3 h. The reaction mixture was cooled to room temperature, and THF was evaporated using rotary vacuum evaporator. The residue was dissolved in hexane and the solution was filtered and the filtrate (polymer solution) was precipitated in methanol. The polymer was allowed to settle down at the bottom. The supernatant liquid was decanted off and the sticky mass was dried under vacuum at room temperature for 12 h. Gravimetric yield: 98%, GPC-MALLS: Mₙ=1400, polydispersity index (PDI)=1.11. ¹H NMR spectrum displayed all the resonances for the methacrylate end-functional PIB indicating quantitative conversion.

### Example 7

### Photopolymerization of Methacrylate End-Functional Polyisobutylene

Methacrylate end-functional PIB (Mₙ=2500, PDI=1.2, 500 mg) was placed in a small sample vial. Irgacure 819 (10 mg, 2 wt %) was added and the sample vial was slightly warmed to prepare a homogeneous solution. A 10 μm fluorinated polyethylene film was first laid down and a thin polyester fiber mesh was placed on top of the plastic film to serve as a spacer. The liquid sample was pipetted onto this assembly and an identical layer of film was placed over the top. In this manner, a reproducible liquid macromonomer layer of 0.912 mm in thickness was achieved and, at the same time, the fiber mesh due to its low volume and thermal mass, does not affect the temperature of the polymerization reaction. The resulting sample sandwich was mounted in the plastic slide holder for UV irradiation. The polymerization was carried out by shining UV light at an intensity of 400 mJ/cm² per minute.

### Example 8

### Photopolymerization of Acrylate End-Functional Polyisobutylene

The networks based on acrylate end-functional PIB was synthesized as follows. Acrylate end-functional PIB (Mₙ=2400, PDI=1.2, 500 mg) was placed in a small sample vial along with Irgacure 651 (10 mg, 2 wt %). The mixture was slightly warmed to prepare a homogenous solution. A 10 μm fluorinated polyethylene film was first laid down and a thin polyester fiber mesh was placed on top of the plastic film to serve as a spacer. The liquid sample was pipetted onto this assembly and an identical layer of film was placed over the top. In this manner, a reproducible liquid macromonomer layer of 0.912 mm in thickness was achieved and, at the same time, the fiber mesh due to its low volume and thermal mass, does not affect the temperature of the polymerization reaction. The resulting sample sandwich was mounted in the plastic slide holder for UV irradiation. The polymerization was carried out by shining UV light at an intensity of 400 mJ/cm² per minute.

### Example 9

### Synthesis of Flexible Hybrid Epoxy Coating

Epoxy end-functional PIB was blended with bisphenol A diglycidyl ether (DGEBA) in different weight proportions such as, epoxy end-functional PIB:DGEBA=5:95, 10:90, 20:80 and 40:60 (wt:wt). The compositions were thermally cured in presence of multifunctional amines. A typical composition and curing procedure is described as follows;

For example, epoxy end-functional PIB (Mₙ=1300, PDI=1.1, 500 mg, 5 wt %) was placed in a small sample vial along with DGEBA (4.5 g, 95 wt %). The mixture was slightly warmed and mixed vigorously to prepare a homogenous solution. To the above solution triethylenetetramine (0.745 g,) was added and mixed using high speed mixer. The resulting blend of epoxy and amine was degassed for 1 h. The samples were then poured into a mould (15 cm×15 cm), which was preheated to 100° C. in a vacuum oven. The samples were then cured at 100° C. for 2 h. Post-cure was conducted at 100° C. for 12 h. After the post-cure, the oven was switched off and allowed to cool slowly to room temperature to avoid crack formation.

Representative flexural strength and modulus data of the networks are listed in Table 1, below.

TABLE 1
Flexural strength and modulus of hybrid epoxy networks

| Epoxy end-functional | Flexural modulus | |
|---|---|---|
| PIB (wt %) | (MPa) | Flexural strength (MPa) |
| 0 | 9000 | 120 |
| 5 | 7350 | 105 |
| 10 | 8000 | 88 |
| 20 | 6400 | 50 |
| 40 | 2600 | 48 |

### Synthesis of Epoxy End-Functional Polyisobutylene

Glycidol has been frequently used in the literature to convert organic halides to epoxides under basic conditions. Preliminary reaction of bromoallyl telechelic PIB with glycidol was attempted using NaH in THF under reflux. The reaction was sluggish yielding 20% end-group conversion in 24 hours. This was attributed to the insolubility of sodium salt of glycidol in THF. The addition of a phase transfer catalyst TBAB increased the homogeneity of the reaction mixture and the substitution was complete in 1.5 hours. ¹H NMR spectroscopy showed the disappearance of peaks at 4.0, 5.7 and 5.75 ppm assigned to the bromomethylene and bromoallylmethine protons and new signals at 4.05, 5.55 and 5.75 ppm assigned to —CH₂OCH₂(CHOCH₂), —CH═CHCH₂OCH₂(CHOCH₂) and —CH═CHCH₂OCH₂(CHOCH₂) appeared indicating quantitative conversion. The —OCH₂(CHOCH₂) exhibited two multiplets at 3.7 and 3.4 ppm whereas the methylene group of the epoxy ring also showed two peaks at 2.8 and 2.6 ppm. In 2D COSY-NMR spectroscopic analysis, cross peaks for 3.7 and 3.4 ppm were obtained with 3.2 ppm indicating that both peaks belong to the methylene group attached to the epoxy moiety. These peak positions were further confirmed by 2D gradient HSQC NMR spectral analysis as both peaks (3.7 and 3.4 ppm) showed connectivity to ═CH—CH₂—OCH₂— (70 ppm) and resonances at 2.8 and 2.6 ppm were correlated to —OCH₂(CHOCH₂) resonated at 45 ppm. The ¹³C NMR spectroscopy supported the conversion by displaying resonances at 132.5 and 121.5 ppm for the olefinic carbons. New signals at 51, 45 and 70 ppm also appeared for —OCH₂(CHOCH₂) and —CH₂OCH₂(CHOCH₂) respectively.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A method of synthesizing a functionalized polymer represented by the structural formula (I) 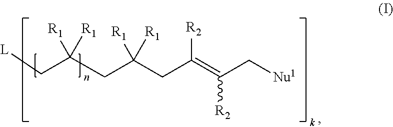
comprising a step of reacting a polymer represented by structural formula (II) 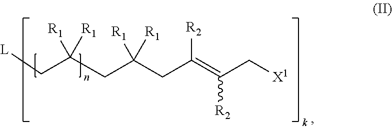
with a compound Nu¹-M to nucleophilically substitute moiety X¹ with moiety Nu¹, wherein:
n is an integer not less than 2;
k is an integer greater than or equal to 1;
L is absent or is an initiator residue;
R₁ for each occasion is independently H or a C1-C4 alkyl, an alkoxy or a substituted or unsubstituted aryl;
R₂ for each occasion is independently H, X², —CH₂X², —CHX²₂, —CX²₃, —C≡N, or —NO₂;
X¹ and X² are, for each occurrence, independently, a halogen;
M is an alkali metal; and
Nu¹ is —Y¹—Y²—R³,
wherein:
Y¹ is absent or is a —NR¹⁰—, —S—, or —O—, wherein R¹⁰ is a C1-C6 alkyl;
Y² is absent or is a C2-C6 alkylene, (—OCH₂CH₂—)₁₋₃, —Si(CH₃)₂—, or a C2-C6 alkylene-O—;
R³ is a C1-C6 alkyl, functionalized by an epoxy, a thriirane, acrylate, methacrylate, cyano acrylate, a vinyloxy or 4,5-dihydrooxazole moiety.

2. The method of claim 1, wherein Y¹ is —O—, Y² is absent or is a C2-C6 alkylene or a C2-C6 alkylene-O—, and R³ is a C1-C6 alkyl, functionalized by an epoxy or a vinyloxy moiety.

3. The method of claim 1, wherein Y¹ is absent, Y² is absent or is a —C1-C6 alkylene or a C1-C6 alkylene-O— or (—OCH₂CH₂—)₁₋₃, and R³ is selected from methacrylate, acrylate, and cyano acrylate.

4. The method of claim 1, wherein Nu¹ is selected from
—O—R^{a}—O—CH═CH₂, wherein R^{a} is a C2-C6 alkylene, and
—R^{b}—OC(O)C(R^{c})═CH₂, wherein R^{b} is absent or is —OCH₂CH₂— and R^{c} is —H, —CN or —CH₃.

5. The method of claim 1, wherein Nu¹ is selected from —O—R^{d}, wherein R^{d} is an epoxy-functionalized C1-C6 alkyl, and 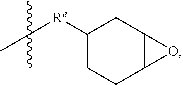
wherein R^{e} is a —O—C2-C6 alkylene, (—OCH₂CH₂—)₁₋₃, or —O—Si(CH₃)₂—CH₂—.

6. The method of claim 1, wherein Nu¹ is selected from 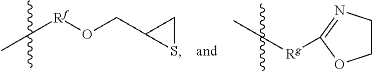
wherein:
R^{f} is a —O—C2-C6 alkylene, or (—OCH₂CH₂—)₁₋₃, and
R^{g} is a —O—C1-C6 alkylene.

7. The method of claim 1, wherein
Y² is absent, or is a —C1-C6 alkylene or (—OCH₂CH₂—)₁₋₃, and
R³ is selected from methacrylate, acrylate, and cyano acrylate.

8. The method of claim 4, wherein Nu¹ is —O—R^{a}—O—CH═CH₂.

9. The method of claim 8, wherein Nu¹ is —O—(CH₂)₂—O—CH═CH₂.

10. The method of claim 4, wherein Nu¹ is —R^{b}—OC(O)C(R^{c})═CH₂.

11. The method of claim 10, wherein Nu¹ is —OC(O)CH═CH₂ or —OC(O)C(CH₃)═CH₂.

12. The method of claim 5, wherein Nu¹ is —O—R^{d}, and R^{d} is a 1,2-epoxy-(C1-C6)alkylene.

13. The method of claim 12, wherein Nu¹ is 1,2-epoxy-1-propoxy group.

14. The method of claim 1, wherein:
R₁ for each occasion is independently H or a C1-C4 alkyl, and
R₂ for each occasion is independently H, X², —CH₂X², —CHX²₂, —CX²₃.

15. The method of claim 12, wherein X¹ is Cl or Br.

16. The method of claim 1 wherein the endcap group represented by the following structural formula 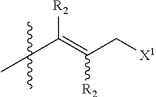
is a chloroallyl group.

17. The method of claim 1 wherein the endcap group represented by the following structural formula 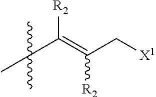
is a bromoallyl group.

18. The method of claim 1, wherein the nucleophilic substitution takes place in the presence of tetra-n-butylammonium bromide (TBAB) or 1,4,7,10,13,16-hexaoxacyclooctadecane (18-crown-6).

19. The method of claim 1, further including a step of producing the polymer represented by structural formula (II) 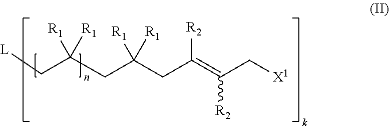
by reacting, in a solvent, a cationic living polymer represented by structural formula (III) 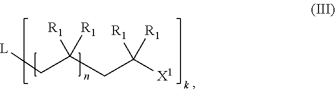
with an optionally substituted conjugated diene represented by structural formula (IV) as an endcapping reagent, in the presence of a Lewis acid, 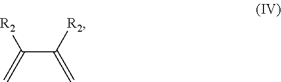
whereby the solvent causes termination by halogenation to be faster than the addition of additional molecules of the conjugated diene, thereby producing the endcapped polymer having a halogenated endcap group.

20. The method of claim 19, further including the step of producing the cationic living polymer represented by structural formula (III) by reacting a cationically polymerizable monomer in the presence of a coinitiator.

21. The method of claim 20, wherein the coinitiator is one or more of BCl₃, TiCl₄, and organo aluminum halides.

22. The method of claim 21, wherein the solvent comprises at least one component having a dielectric constant less than about 9.

23. The method of claim 22, wherein the solvent is selected from one or more of hexane, cyclohexane, methylcyclohexane, methylchloride, n-butyl chloride, dichloromethane, toluene, and chloroform.

24. The method of claim 1, wherein X¹ is Br.

25. The method of claim 1, wherein L is 5-tert-butyl-dicumyl, 1,3,5-tri-cumyl, 2,4,4,6-tetramethylheptyl, or 2,5-dimethylhex-3-en-yl.

26. The method of claim 1, wherein k is 2, and L is represented by the following structural formula

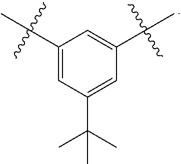

27. The method of claim 1, wherein the polymer of formula (II) is represented by the following structural formula:

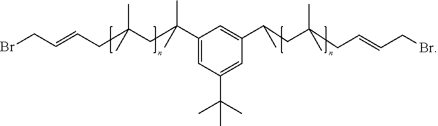

28. The method of claim 1, wherein:
Nu¹ is represented by the following structural formula 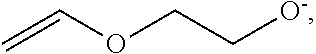
and
the polymer of formula (I) is represented by structural formula (V):

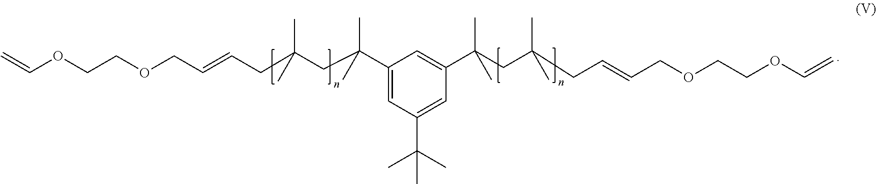

29. The method of claim 1, wherein:
Nu¹ is represented by the following structural formula 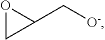
and
the polymer of formula (I) is represented by structural formula (Vi):

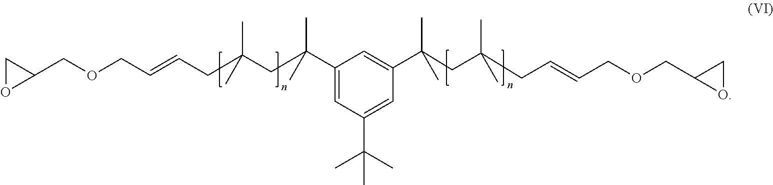

30. The method of claim 1, wherein:
Nu¹ is represented by the following structural formula 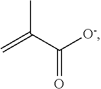
and
the polymer of formula (I) is represented by structural formula (VII):

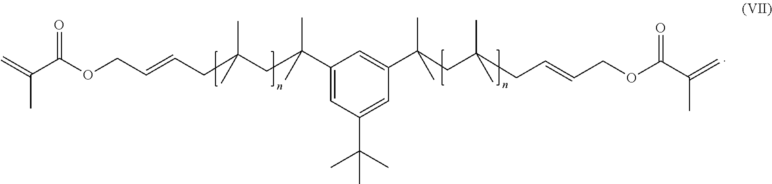

31. The method of claim 1, wherein:
Nu¹ is represented by the following structural formula 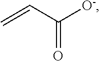
and
the polymer of formula (I) is represented by structural formula (VIII):

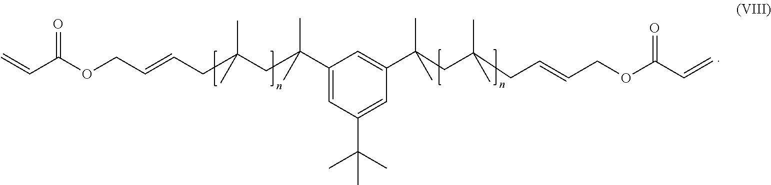

32. A functionalized polymer represented by structural formula (I): 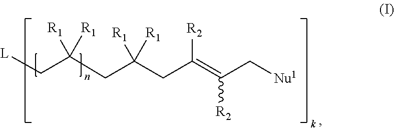
wherein:
n is an integer not less than 2;
k is an integer greater than or equal to 1;
L is absent or is an initiator residue;
R₁ for each occasion is independently H or a C1-C4 alkyl, an alkoxy or a substituted or unsubstituted aryl;
R₂ for each occasion is independently H, X², —CH₂X², —CHX²₂, —CX²₃, —CN, or —NO₂;
X¹ and X² are, for each occurrence, independently, a halogen;
M is an alkali metal; and
Nu¹ is —Y¹—Y²—R³,
wherein:
Y¹ is absent or is a —NR¹⁰—, —S—, or —O—, wherein R¹⁰ is a C1-C6 alkyl;
Y² is absent or is a C₂-C₆ alkylene, (—OCH₂CH₂—)₁₋₃, —Si(CH₃)₂—, or a C2-C6 alkylene-O—;
R³ is a C₁-C₆ alkyl, functionalized by an epoxy, a thriirane, acrylate, methacrylate, cyano acrylate, a vinyloxy or 4,5-dihydrooxazole moiety.

33. The polymer of claim 32, wherein Y¹ is —O—, Y² is absent or is a C2-C6 alkylene or a C2-C6 alkylene-O—, and R³ is a C1-C6 alkyl, functionalized by an epoxy or a vinyloxy moiety.

34. The polymer of claim 33, wherein Y¹ is absent, Y² is absent or is a —C1-C6 alkylene or a C1-C6 alkylene-O— or (—OCH₂CH₂—)₁₋₃, and R³ is selected from methacrylate, acrylate, and cyano acrylate.

35. The polymer of claim 32, wherein Nu¹ is selected from
—O—R^{a}—O—CH═CH₂, wherein R^{a} is a C2-C6 alkylene, and
—R^{b}—OC(O)C(R^{c})═CH₂, wherein R^{b} is absent or is —OCH₂CH₂— and R^{c} is —H, —CN or —CH₃.

36. The polymer of claim 32, wherein Nu¹ is selected from
—O—R^{d}, wherein R^{d} is an epoxy-functionalized C1-C6 alkyl, and 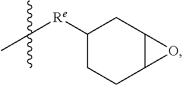
wherein R^{e} is a —O—C2-C6 alkylene, (—OCH₂CH₂—)₁₋₃, or —O—Si(CH₃)₂—CH₂—.

37. The polymer of claim 32, wherein Nu¹ is selected from 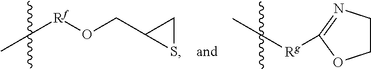
wherein:
R^{f} is a —O—C2-C6 alkylene, or (—OCH₂CH₂—)₁₋₃, and
R⁹ is a —-C1-C6 alkylene.

38. The polymer of claim 32, wherein
Y² is absent, or is a —C1-C6 alkylene or (—OCH₂CH₂—)₁₋₃, and
R³ is selected from methacrylate, acrylate, and cyano acrylate.

39. The polymer of claim 35, wherein Nu¹ is —O—R^{a}—O—CH═CH₂.

40. The polymer of claim 39, wherein Nu¹ is —O—(CH₂)₂—O—CH═CH₂.

41. The polymer of claim 35, wherein Nu¹ is —R^{b}—OC(O)C(R^{c})═CH₂.

42. The polymer of claim 41, wherein Nu¹ is —OC(O)CH═CH₂ or —OC(O)C(CH₃)═CH₂.

43. The polymer of claim 36, wherein Nu¹ is —O—R^{d}, and R^{d} is a 1,2-epoxy-(C1-C6)alkylene.

44. The polymer of claim 43, wherein Nu¹ is 1,2-epoxy-1-propoxy group.

45. The polymer of claim 32, wherein:
R₁ for each occasion is independently H or a C1-C4 alkyl, and
R₂ for each occasion is independently H, X², —CH₂X², —CHX²₂, —CX²₃.

46. The polymer of claim 45, wherein X² is Cl or Br.

47. The polymer of claim 32, wherein X² is Br.

48. The polymer of claim 32, wherein L is 5-tert-butyl-dicumyl, 1,3,5-tri-cumyl, 2,4,4,6-tetramethylheptyl, 2,5-dimethylhex-3-en-yl.

49. The polymer of claim 32, wherein k is 2, and L is represented by the following structural formula

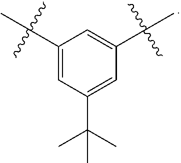

50. The polymer of claim 32, represented by structural formula (V):

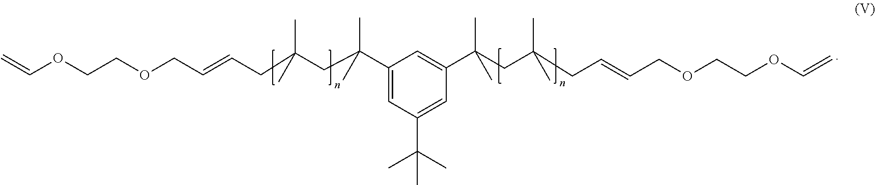

51. The polymer of claim 32, represented by structural formula (VI):

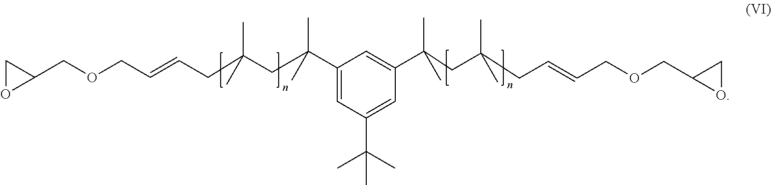

52. The polymer of claim 32, represented by structural formula (VII):

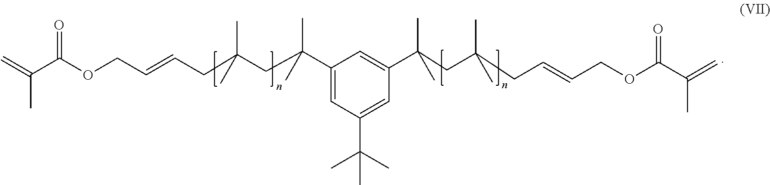

53. The polymer of claim 32, represented by structural formula (VIII): [chemical structure]
